(19) United States
(12) Patent Application Publication
(10) Pub. No.: US 2021/0330576 A1
(43) Pub. Date: Oct. 28, 2021

(54) Magic Skin Repair Serum

(71) Applicant: Brittnei Blackwell, Brooklyn, NY (US)

(72) Inventor: Brittnei Blackwell, Brooklyn, NY (US)

(21) Appl. No.: 16/859,609

(22) Filed: Apr. 27, 2020

(51) Int. Cl. A61K 8/9789; A61K 8/9794; A61K 8/67; A61K 8/06; A61Q 19/08

(52) U.S. Cl. CPC A61K 8/9789; A61K 8/9794; A61K 8/675; A61K 2800/5922; A61Q 19/08; A61K 2800/10; A61K 8/062

(57) ABSTRACT

The present invention discloses an advance enhanced stability, enhanced solubility and an enhanced photoprotective effect by proposing an improved process by presenting a technique where the skin care products inform of serum is presented. The proposed method provides compositions with necessary ingredients including but not limited to Organic rose hip oil, organic wheat germ, organic Gotu kola, organic onion seed oil and vitamins such as A, B3, B5 and E containing cosmetics used to address age, wrinkles, stretch marks, dark spots, spider veins, razor bumps, ingrown hair, loose and soggy skin and environmental induced damage.

### BACKGROUND

### Field of the Invention

The present invention relates to skincare. More specifically, the present invention relates to the field of skincare product designed to prevent heal wrinkles, stretch marks and scars in sun-damaged or aging skin caused on body and face.

### Description of the Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Although compositions for skin care products operate through a variety of mechanisms, most provide only temporary moisture retention for improved hydration, transitory improvement in appearance and/or superficial relief of skin integrity.

Human skin is composed of three primary layers: the epidermis, dermis, and hypodermis. Each of these layers contains specific cell populations embedded within a complex physical and biochemical matrix, thereby providing the mechanical and functional organization necessary for the skin to maintain barrier function. It is well-known that the epidermis sloughs millions of cells every day, creating an enormous demand for replacement cells. Central to this process, which operates continuously throughout an organism's lifetime, are the various populations of progenitors and stem cells and the associated fibroblasts which provide biomechanical and trophic support. Progenitors and stem cells proliferate and differentiate as part of the natural repair and regeneration mechanism in the body, in order to meet this continual demand for new cells in the skin. Fibroblasts, which populate the dermis, provide vital trophic support, while producing important protein components of the skin.

Aging is a multifactorial phenomenon. The aging of the skin is mainly the result of one's genetic predisposition (known as chronological aging) and one's physiological reaction to environmental stresses (known as actinic aging). Chronological aging is largely genetically driven and appears to be largely linked to a reduction in anti-oxidant production. Actinic aging seems to be skin specific and is defined as the effect of the external environment on the skin's biological response. The skin response to actinic aging, which may be caused by sun and pollution exposure as well as smoking, is typically associated with a lack of normal hydration, apparition of telangiectasia, sagging of the skin, and the appearance of fine lines and wrinkles.

Conventional anti-aging skin products target a single or a few causes of aging. There are multiple inventions that have been proposed in prior art regarding skin care products. For instance, a skin care compositions containing combinations of natural ingredients bearing U.S. Pat. No. 9,289,375B2 is issued to STEMTECH INTERNATIONAL Inc. The patent described herein are various natural ingredients for skin-related applications. Individual compounds provide beneficial effects for improved collagen production, fibroblast proliferation, antioxidant protection and free radical inhibition. Combinations of these individual compounds exhibit synergistic effects, leading to dramatic improvements in skin moisture levels, wrinkle reduction, and elasticity. Various examples of combined ingredients are provided, which can be applied in methods related to conditioning the skin, and in cosmetic formulations for improved aesthetic appearance.

Another patent on Cosmetic composition to accelerate repair of functional wrinkles bearing U.S. Pat. No. 8,025,907B2 is issued to BELFER COSMETICS LLC. The patent is on a cosmetic composition consists of a combination of peptides that regenerate the dermal matrix and a rapid acting muscle relaxant derived from the extract of the plant, Acmella oleracea. By limiting cutaneous deformation caused by the contraction of facial muscles the extract increases the efficiency of the peptides in reducing expression lines.

Another patent on Skin care compositions and methods of use thereof bearing Canadian patent 2,749,750C is issued to IMMANENCE INTEGRALE DERMO CORRECTION Inc. The patent describes a topical composition comprising at least 10 isolated active agents which each have at least one activity selected from the group consisting of anti-oxidant activity, anti-matrix metalloproteinase (MPP) activity, anti-elastase activity, anti-inflammation activity, anti-irritation activity, microcirculation support activity, collagen synthesis activity, energy production activity, oxygenation activity, DNA protection and repair activity, cell anchorage suppport activity, dermal-epidermal cohesion support activity, cellular renewal activity, synthesis and/or protection of glycosaminoglycans activity, prevention and/or repair and/or elimination of damaged proteins activity, protection of skin immune function activity, hydration activity, modulation of skin pigmentation activity, and anti-cellulite activity, the composition possessing at least five of the foregoing activities. Methods of use thereof.

A Skin care composition and treatment bearing Japanese patent 2,013,224323A is issued to Andrew J Nixon. The patent provides a therapeutic agent or skin care product for photoaged skin. (A) a carrier substrate; (b) a conditioned cell medium containing one or more cultured skin materials synthesized and secreted from cultured keratinocytes and fibroblasts;) Silicone based penetration enhancers. A composition comprising a growth substance synthesized from cultured cells from the skin. Skin cells such as keratinocytes and dermal fibroblasts are cultured in vitro in a cell culture medium. In the course of the culture, the cultured cells synthesize substances and secrete them into the cell culture medium. This medium containing the substance is collected and incorporated into a pharmaceutical or cosmetic preparation for treating an individual.

Another patent on Use of cellular extracts for skin rejuvenation bearing U.S. Pat. No. 8,557,295B2 is issued to Regenics AS. The patent is on methods and agents for improving cosmetic appearance, for promoting, improving or restoring health of cells and tissues, preferably skin, and more preferably, for restoring aged or damaged skin to a healthy appearance. In preferred embodiments, the methods and agents comprise active extracts produced from fish eggs. The invention further provides processes for making active fish egg extracts.

Another patent on Topical skin care formulations bearing U.S. Pat. No. 8,1781,06B2 is issued to Kay Mary Inc. The patent discloses a topical skin care composition comprising: (a) a combination of the following extracts: Malpighia punicifolia (acerola) extract; Argania spinosa (argan) extract; Myrciaria dubia (camu camu) extract; Punica granatum (pomegrannate) extract; Pinus sylvestris extract; Terminalia ferdinandiana (kakadu plum) extract; Linum usitatissimum (linseed) extract; Ribes nigrum (black current) extract; Secale cereale (rye) extract; algae extract; and yeast extract; and (b) a dermatologically acceptable vehicle.

A Cosmetic composition bearing U.S. Pat. No. 2,019,0099361A1 is issued to Kay Mary Inc. The patent discusses compositions and methods for their use that can be used in cosmetic applications. The composition can include an effective amount of a Centella asiatica stem cells to reduce the activity of hyaluronidase in skin, an effective amount of tetradecyl aminobutyroylvalylamino butyric urea trifluoroacetate or Alpinia galanga leaf extract to promote the production of hyaluronic acid in skin, an effective amount of tripeptide-1 to promote the production of fibronectin and laminin in skin, and a dermatologically acceptable vehicle.

A number of skin care products and skin care treatments have been developed to improve the appearance of the skin. In addition, various therapeutic agents have been developed to treat chronic skin disorders such as acne, precancerous lesions, scars, pigmentation abnormalities, and epilepsy. There are multiple inventions that have been seen in prior art however Regardless of the approach, these methods generally are inadequate to facilitate the user when skin care is required for multiple health conditions.

Looking at the prior art in similar field, there have been pharmaceutical (such as dermatological and ophthamological) and cosmetic products previously developed and marketed which contain these solutions are found to be limited and restricted to their conventional methodologies and techniques. The current methodology provides a new method for an enhanced methodology where a new approach of pairing skin care product is proposed. As described herein, the inventor has discovered new and useful compositions derived from natural ingredients in many of these plants act by supporting the proliferation, migration and differentiation of skin progenitor and stem cells and/or fibroblasts. These natural ingredients can be applied topically in a blended mixture (including but not limited to Organic rose hip oil, organic wheat germ, organic Gotu kola and organic onion seed oil), proved to have a significant effect on moisture retention in the skin, skin elasticity, as well as wrinkle reductions.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved method of skin repair serum to treat multiple skin problems including but not limited to dark spots, wrinkles, stretch marks, scars, fine lines, acne, razor bumps, ingrown hair, keloid scars, spider veins and loose skin.

It is further the desirable object of the present invention is to provide a novel and improved form of method of stabilization of vitamins with Ascorbic acid and mineral salts of ascorbic acid mixtures together with oils in the dry phase.

More particularly it is a primary object of the invention to provide a new method for the stabilization and increased shelf life of an skin serum, mineral salts of ascorbic acid and vitamins such as A, B3, B5 and E containing cosmetics used to address radical induced damage.

Another object of the invention is to provide a method of improving skin appearance in a subject, including providing a quantity of a purified composition, wherein the composition includes one or more of the following components selected from the group including: Organic rose hip oil, organic wheat germ, organic Gotu kola and organic onion seed oil.

It is moreover the objective of the invention for preventing or reducing a skin aging sign or a skin condition or disorder in a subject comprising applying an effective amount of the composition of the present invention on the skin of the subject, whereby the skin aging sign or a skin condition or disorder is prevented or reduced.

It is also the object of the invention to enhance the provided use of a composition of the invention for improving skin appearance or for the manufacture of a medicament for improving skin appearance.

It is further the objective of the invention to provide a new, more economical and stable topical composition of dermatological compositions for treatment of skin to address radical-induced damage.

Still yet another object of the present invention is to provide a new and improved stabilized ascorbic acid (vitamin C), mineral salts of ascorbic acid and derivatives of ascorbic acid in cosmetic dermatological compositions for treatment of skin to address radical-induced damage, which provides in the apparatuses and methods of the prior art some of the advantages thereof, while simultaneously overcoming some of the disadvantages normally associated therewith.

Still yet another object of the present invention is to provide a new and improved methodology, which provides in the apparatuses and methods of the prior art some of the advantages thereof, while simultaneously overcoming some of the disadvantages normally associated therewith.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Many factors contribute to skin aging such as the actual age of a person, the amount of exposure to environmental factors (e.g., sun light, pollution, chemicals, smoke, etc.), and how well a person has taken care of their skin. In particular, skin aging concerns two processes—intrinsic aging, which is related to the natural aging process and genetic influences, and extrinsic, or accumulated damage due to environmental factors such as sun exposure. This combination of factors eventually leads to visible signs of aging, and over time these signs progress through three stages—early, moderate and advanced.

The early signs of skin aging include the first stages of stretch marks, scars, visible fine lines, especially around the eyes, and the beginning of uneven skin tone. Cell turnover begins to slow, and this can have a dulling effect on the complexion. Collagen and elastin—while still healthy—can start to suffer early damage, leaving skin slightly less resilient. If the matrix is left unprotected, wrinkles that are forming underneath the surface of the skin will eventually become more noticeable due to damage in the dermal layer. Eyes can occasionally look puffy, and pores appear slightly more noticeable. Typically, this occurs in an age range of about 25 to 35 years of age.

The moderate signs of skin aging include more pronounced expression lines around the eyes, the mouth and on the forehead. Underneath the eyes dark circles can become more noticeable. The skin's support structure becomes weaker as less collagen is produced, and elastin fibers begin to lose their ability to “snap” back. Skin loses vital moisture more easily, and dark spots can become more of an issue. Fine lines on the neck can become more visible, and “marionette” lines on either side of the mouth can begin to appear. More significant age spots begin to surface, eyes may look tired more often, and pores appear larger. This typically occurs in an age range of about 35 to 50 years of age.

The advanced signs of skin aging include “static” deep lines and wrinkles that are visible even when the face is at rest. The supporting structure of collagen and elastin is severely compromised and skin sagging, especially in the cheek and jawline areas, becomes evident. The neck shows signs of cumulative damage, with the skin becoming loose and marked by horizontal wrinkles called “tree rings.” Dark spots become more prominent, and the eye area can show noticeable crepiness, sagging, puffiness and more pronounced dark circles in addition to a “drooping” upper eyelid. Skin loses its youthful volume and lift due to a loss of natural cushioning, and skin dryness is more pronounced as the external barrier is compromised, oil production slows and internal moisture levels drop. Cell turnover slows dramatically, and dead skin cells remain on the skin's surface which can dull the complexion and make pores more noticeable. The thickness of the skin is also impacted, and as it becomes thinner it's more easily irritated. Typically this occurs in an age range of above 50 years of age.

Current products on the market either do not effectively address ageing skin or have skin irritating effects. The inventors, however, have discovered a unique combination of ingredients that work in a symbiotic relationship with one another to effectively address the signs of early, moderate, and advanced skin ageing.

The present invention is directed to an improved system and procedure for a stabilized cosmetic dermatological composition for treatment of skin to address multiple types of skin damages coupled with skin meditation approach.

The present invention discloses a new method for the stabilization and enhanced formula wellness product with ingredients including but not limited to Organic rose hip oil, organic wheat germ, organic Gotu kola and organic onion seed oil to enhance the product effectiveness.

The invention as per its preferred embodiments presents a new method where the skin care products are coupled with meditation technique. The meditation technique works internally while skin care products work in their own way to enhance the structure of skin.

As used herein, the term “cosmetic agent” means any substance, as well as any component thereof, intended to be rubbed, poured, sprinkled, sprayed, introduced into, or otherwise applied to a human skin or any part thereof for purposes of cleansing, beautifying, promoting attractiveness, altering the appearance, or combinations thereof. Cosmetic agents include, but are not limited to, substances that are Generally Recognized as Safe (GRAS) by the U.S. Food and Drug Administration, food additives, and materials used in non-cosmetic consumer products including over-the-counter medications. In some embodiments, cosmetic agents may be incorporated in a cosmetic composition comprising a dermatologically acceptable carrier suitable for topical application to skin.

In certain embodiments, compositions of the present invention can decrease the amount of internal oxidation and/or external oxidative damage in a cell. In other aspects, the compositions can increase collagen synthesis in a cell. The compositions can also reduce skin inflammation, such as by reducing inflammatory cytokine production in a cell. Non-limiting examples of such cells include human epidermal keratinocyte, human fibroblast dermal cell, human melanocytes, three dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

Also disclosed is a method of lightening skin or evening skin tone comprising applying the compositions of the present invention to the skin. The method can further comprise identify a person in need of lightening skin or evening skin tone. The methods can further include inhibiting melanogenesis in a skin cell, inhibiting tyrosinase or tyrosinase synthesis in a skin cell, or inhibiting melanin transport to keratinocytes in a skin cell. The composition can act as an alpha melanin stimulatory hormone antagonist. The composition can even out pigmentation of the skin. In non-limiting aspect, lightening skin can include reducing the appearance of an age spot, a skin discoloration, or a freckle by topical application of the composition to skin having an age spot, skin discoloration, a freckle, etc.

Also disclosed is a method of treating hyperpigmentation comprising applying the compositions of the present invention to the skin. The method can also comprise identifying a person in need of treating hyperpigmentation. Additional methods contemplated by the inventor include methods for reducing the appearance of an age spot, a skin discoloration, or a freckle, reducing or preventing the appearance of fine lines or wrinkles in skin, or increasing the firmness of skin.

In a particular embodiment there is disclosed a method of reducing the appearance of a skin condition comprising topically applying any one of the compositions described in this specification to the skin condition, wherein topical application of the composition to skin condition reduces the appearance of the skin condition. The skin condition can be any one of those described in this specification. In particular aspects, the skin condition is a fine line or wrinkle, uneven skin tone, or an age spot. The skin condition can be located on facial skin, arm skin, leg skin, chest skin, abdomen skin, back skin etc.

In yet another embodiment there is disclosed a method of increasing the firmness of skin comprising topically applying any one of the compositions described in this specification to skin in need thereof (a non-limiting example of which can be sagging skin, aged skin, skin that has reduced elasticity, skin that has skin cells having inadequate amounts of collage, fibronectin, or laminin or all of such proteins, etc.), wherein topical application of the composition to skin increases the firmness of skin. The composition can be used on facial skin, arm skin, leg skin, chest skin, abdomen skin, back skin, etc.

In still another embodiment there is disclosed a method of increasing collagen, fibronectin, or laminin production in a skin cell comprising topically applying any one of the compositions disclosed in this specification to a skin cell that is in need of collagen, fibronectin, or laminin production, wherein the topical application of the composition to the skin cell increases collagen, fibronectin, or laminin production in the skin cell. Typical skin cells can be found in skin that is aged, damaged by UV radiation, damaged by environmental factors, wrinkles, etc. In certain aspects, collagen, fibronectin, and laminin production are increased in the skin cell.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: A stable composition comprising:
an individual packaging of the active ingredients together with a heterogeneous mixture of Organic rose hip oil, organic wheat germ, organic Gotu kola and organic onion seed oil
other vitamins such as Vitamin, Vitamin B3, Vitamin B5, Vitamin and small peptides are preserved in the dry mixture of above ingredients.

II: A tropical composition where:
the topical skin care composition of claim 1, wherein the composition is formulated as an oil-in-water emulsion;
the topical skin care composition of claim 1, wherein the composition is formulated as a serum;
our unique serum diminishes stretch marks and scars as well;
a method of reducing the appearance of a skin condition comprising topically applying the composition of claim 1 to the skin condition, wherein topical application of the composition to skin condition reduces the appearance of the skin condition, wherein the skin condition is a fine line or wrinkle, uneven skin tone, or an age spot;
the method of claim 1, wherein the skin condition is located on facial skin;
a method of increasing the firmness of skin comprising topically applying the composition of claim 1 to skin in need thereof, wherein topical application of the composition to skin increases the firmness of skin.
